(19) United States
(12) Patent Application Publication
(10) Pub. No.: US 2023/0266267 A1
(43) Pub. Date: Aug. 24, 2023

(54) SARS-CoV-2 BIOSENSOR UTILIZING A PHOSPHATASE REPORTER

(71) Applicant: UNIVERSITY OF SOUTHERN CALIFORNIA, Los Angeles, CA (US)

(72) Inventors: Moh Amer, Los Angeles, CA (US); Mingrui Chen, Los Angeles, CA (US); Chongwu Zhou, Los Angeles, CA (US)

(21) Appl. No.: 18/112,695

(22) Filed: Feb. 22, 2023

Related U.S. Application Data
(60) Provisional application No. 63/312,574, filed on Feb. 22, 2022.

(51) Int. Cl. G01N 27/414; G01N 33/543; B82Y 15/00

(52) U.S. Cl. CPC G01N 27/4145; G01N 27/4146; G01N 33/54373; G01N 27/414; B82Y 15/00

(57) ABSTRACT

A biosensor capable of detecting both the SARS-CoV-2 S1 spike protein antigen and the SARS-CoV-2 spike protein IgG antibody is disclosed. In various embodiments, the biosensor is configured with an array of solution-gated nanoribbon FETs, wherein each nanoribbon comprises indium oxide (In₂O₃). In various aspects, the biosensor is fabricated using a scalable and cost-efficient lithography-free process comprising shadow masking.

### CROSS-REFERENCE TO RELATED APPLICATIONS

This application claims priority to and the benefit of U.S. Provisional Patent Application Ser. No. 63/312,574 filed Feb. 22, 2022 entitled “HIGHLY SENSITIVE, SCALABLE AND RAPID SARS-CoV-2 BIOSENSOR BASED ON INDIUM OXIDE (In₂O₃) NANORIBBON TRANSISTORS AND PHOSPHATASE,” the disclosure of which is incorporated herein by reference in its entirety for all purposes.

### FIELD

The present disclosure generally relates to biosensors, and in particular to nanoribbon field-effect transistor (FET) devices capable of detecting S1 antigen of SARS-CoV-2 and SARS-CoV-2 S1 protein IgG antibody, and methods of using the devices.

### BACKGROUND

On the 11 Mar. 2020, the World Health Organization (WHO) declared Coronavirus disease 2019 (COVID-19) outbreak, caused by the severe acute respiratory syndrome coronavirus (SARS-CoV-2), as a pandemic. SARS-CoV-2 can cause severe respiratory distress, and as of November 2021, the cumulative number of infected people was over 250 million, with over 5 million deaths globally. Owing to the high infectivity of this virus and lack of specific treatments for COVID-19, early diagnosis of COVID-19 in patients is an essential part of pandemic prevention and control.

Thus far, based on previously established laboratory protocols, diagnosis of COVID-19 depends mainly on nucleic acid amplification, namely, real-time reverse transcription-polymerase chain reaction (RT-PCR). Although this method is sensitive and shows good specificity for COVID-19 detection, this technique has a long processing time (usually 4-5 hours) and requires advanced instruments, expensive reagents, and skilled technicians.

Therefore, highly sensitive, scalable and cost-efficient biosensors and methods are still needed for rapid detection of SARS-CoV-2. New sensors and methods are also needed that can detect SARS-CoV-2 human antibodies, such as for identifying past infections and for assessing the effectiveness of vaccines. New detection biosensors and methods are particularly needed for developing countries carrying a heavy economic burden caused by the COVID-19 pandemic.

### SUMMARY

In accordance with various embodiments of the present disclosure, a biosensor capable of detecting both SARS-CoV-2 S1 antigen and SARS-CoV-2 spike protein IgG antibody is described. The present biosensor is based on an improved indium oxide (In₂O₃) nanoribbon field-effect transistor (FET) biosensor platform fabricated using a scalable and cost efficient lithography free process utilizing shadow masks.

In various embodiments, a biosensor capable of detecting both SARS-CoV-2 S1 antigen and SARS-CoV-2 spike protein IgG antibody comprises a FET device configured with source and drain electrodes electrically connected by a nanoribbon comprising indium oxide In₂O₃. In various embodiments, the biosensor is configured as a solution-gated nanoribbon FET further comprising a gate electrode. In various embodiments, a biosensor capable of detecting both SARS-CoV-2 antigen and SARS-CoV-2 spike protein IgG antibody comprises a FET device configured with source and drain electrodes electrically connected by a nanoribbon consisting of only indium oxide In₂O₃.

In various aspects, the FET biosensor in accordance with the present disclosure consists of an indium oxide (In₂O₃) channel and a newly developed stable phosphatase enzyme reporter. The biosensors herein apply phosphatase as an enzyme reporter, resulting in greater stability than the widely used urease in FET based biosensors.

In various embodiments, In₂O₃ biosensors in accordance with the present disclosure reliably detect the S1 antigen of SARS-CoV-2 in both phosphate-buffered saline (PBS) buffer and universal transport medium (UTM) with similar limits of detection (LoD) of 100 fg/mL. Further, In₂O₃ biosensors in accordance with the present disclosure were able to reliably detect the S1 protein IgG antibody in both PBS and human whole blood (WB) with similar LoD of 1 pg/mL.

The similar detection results obtained in different mediums indicate the capability of In₂O₃ biosensors in accordance with the present disclosure to eliminate the interference of salts, cells, and other particles in detected specimens. Taken together, the biosensors disclosed herein are highly sensitive and scalable, showing good potential for clinical SARS-CoV-2 diagnosis. The findings disclosed herein shed light on a new generation of biosensors for rapid and highly sensitive COVID-19 screening that will assist in managing the ongoing COVID-19 pandemic.

In various embodiments, a method of detecting the presence of, or a concentration of, an antigen in a sample comprises fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibody being specific to the antigen, and wherein the antigen binds to the immobilized capture antibody; fluidically contacting biotinylated secondary antibodies with the array of In₂O₃ FET devices of the biosensor, the biotinylated secondary antibodies being specific to the antigen, wherein the biotinylated secondary antibody binds to the antigen previously bound to the immobilized capture antibody; fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation; fluidically contacting alkaline phosphatase substrate with the array of In₂O₃ FET devices of the biosensor, the alkaline phosphatase substrate being capable of enzymatic cleavage by the alkaline phosphatase, wherein the alkaline phosphatase cleaves the alkaline phosphatase substrate, producing protons and a pH change in the liquid medium; and detecting an amperometric signal from the biosensor as a result of the pH change in the liquid medium, indicating a presence of the antigen in the sample.

In various embodiments, the pH change in the liquid medium comprises a pH decrease resulting in an increase in conductivity of each In₂O₃ nanoribbon.

In various embodiments, the method further comprises determining a concentration of the antigen in the sample by interpolating the amperometric signal thus detected on a calibration curve that relates amperometric signal to antigen concentration.

In various embodiments, the calibration curve comprises an x/y plot of percent current change in the biosensor ((ΔI/I₀ (%)) versus antigen concentration.

In various embodiments, the antigen comprises SARS-CoV-2 S1 antigen.

In various embodiments, the alkaline phosphatase substrate comprises disodium p-nitrophenyl phosphate.

In various embodiments, the biosensor further comprises a solution gate electrode in fluidic contact with the liquid medium such that each In₂O₃ FET device is configured as a solution-gated FET.

In various embodiments, the method further comprises applying a liquid gate voltage to the solution gate electrode prior to detecting an amperometric signal comprising a current or change in current in the biosensor.

In various embodiments, each In₂O₃ nanoribbon is characterized by a channel length about 500 μm, a channel width of about 25 μm, and a channel thickness of about 18 nm.

In various embodiments, a method of detecting the presence of, or a concentration of, an antibody in a sample specific to an antigen comprises fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibody being specific to and having the antigen bound thereto, and wherein the antibodies in the sample bind to the antigens bound to the immobilized capture antibodies; fluidically contacting biotinylated secondary antibodies with the array of In₂O₃ FET devices of the biosensor, the biotinylated secondary antibodies comprising anti-human antibody to the antigen, wherein the biotinylated secondary antibodies bind to the antibodies previously bound to the antigens immobilized on the capture antibodies; fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation; fluidically contacting alkaline phosphatase substrate with the array of In₂O₃ FET devices of the biosensor, the alkaline phosphatase substrate capable of enzymatic cleavage by the alkaline phosphatase, wherein the alkaline phosphatase cleaves the alkaline phosphatase substrate, producing protons and a pH change in the liquid medium; and detecting an amperometric signal from the biosensor as a result of the pH change in the liquid medium, indicating a presence of the antibodies in the sample.

In various embodiments, the antibody in the sample comprises SARS-CoV-2 spike protein IgG antibody and the antigen to which the antibody is specific to comprises SARS-CoV-2 S1 antigen.

In various embodiments, the method further comprises determining a concentration of the antibodies in the sample by interpolating the amperometric signal thus detected on a calibration curve that relates amperometric signal to antibody concentration.

In various embodiments, the calibration curve comprises an x/y plot of percent current change in the biosensor ((ΔI/I₀ (%)) versus antibody concentration.

In various embodiments, the alkaline phosphatase substrate comprises disodium p-nitrophenyl phosphate.

In various embodiments, the biosensor further comprises a solution gate electrode in fluidic contact with the liquid medium such that each In₂O₃ FET device is configured as a solution-gated FET.

In various embodiments, the method further comprises applying a liquid gate voltage to the solution gate electrode prior to detecting an amperometric signal comprising a current or change in current in the biosensor.

In various embodiments, each In₂O₃ nanoribbon is characterized by a channel length about 500 μm, a channel width of about 25 μm, and a channel thickness of about 18 nm.

In various embodiments, the pH change in the liquid medium comprises a pH decrease resulting in an increase in conductivity of each In₂O₃ nanoribbon.

### BRIEF DESCRIPTION OF THE DRAWING FIGURES

The subject matter is pointed out with particularity and claimed distinctly in the concluding portion of the specification. A more complete understanding, however, may best be obtained by referring to the detailed description and claims when considered in connection with the following drawing figures:

FIG. 1A illustrates the biological structure of SARS-CoV-2 virus, showing the various types of proteins;

FIG. 1B is a photograph of a 3-inch wafer fabricated in accordance with the present disclosure, comprising a plurality of twenty eight (28) In₂O₃ nanoribbon FET devices fabricated on chips of four FET devices each, with the chips arranged in a 4×7 array;

FIG. 1C is a magnified image of a channel region of one In₂O₃ nanoribbon FET device on the wafer of FIG. 1B, with the scale bar measuring 200 μm;

FIG. 1D is an Atomic Force Microscopy (AFM) image of an 18 nm thick In₂O₃ nanoribbon within a single In₂O₃ nanoribbon FET device of the wafer of FIG. 1B;

FIG. 1E is a graph of nine drain current versus back gate voltage curves obtained by analyzing nine separate In₂O₃ nanoribbon FET devices on the wafer of FIG. 1B with the data presented linear and logarithmically;

FIG. 1F schematically illustrates an embodiment of a functionalized In₂O₃ nanoribbon FET device configured to detect SARS-CoV-2 S1 protein, in accordance with the present disclosure;

FIG. 2A illustrates the alkaline phosphatase enzymatic cleavage reaction used in the present sensing platform, wherein the production of protons from the reaction causes a measurable change in pH;

FIG. 2B is a plot of pH over time once a substrate solution comprising the reactants of FIG. 2A is mixed with a phosphatase solution and the reaction of FIG. 2A commences;

FIG. 2C is a graph of linear and logarithmic drain current versus liquid gate current plots, with drain voltage fixed at 1 volt;

FIG. 2D illustrates a plot of current versus solution pH (inset) and real-time responses obtained from an In₂O₃ nanoribbon device exposed to commercial buffer solutions with pH from to 4;

FIG. 2E schematically illustrates an embodiment of a In₂O₃ nanoribbon FET device configured for positive control measurements;

FIG. 2F is a plot of current versus time from the positive control setup of FIG. 2E;

FIG. 3A is a plot of real-time responses monitored at 100 fg/mL, 1 pg/mL, 10 pg/mL, 100 pg/mL and 1 ng/mL concentrations of S1 proteins in PBS;

FIG. 3B is a plot of real-time responses monitored at 100 fg/mL, 1 pg/mL, 10 pg/mL, 100 pg/mL and 1 ng/mL concentrations of S1 proteins in UTM;

FIG. 3C is a graph showing a comparison between the plots of normalized responses at each S1 protein concentration in PBS and UTM, in nanograms per milliliter, with the graph usable as a calibration curve for interpolating an S1 protein concentration given a measured response;

FIG. 3D is a plot of real-time responses monitored using four biosensors from four fabrication batches at 10 pg/mL of S1 protein, showing the reproducibility of the present technique;

FIG. 4A schematically illustrates an embodiment of a In₂O₃ nanoribbon FET device configured to detect SARS-CoV-2 S1 protein IgG antibody, in accordance with the present disclosure;

FIG. 4B is a plot of real-time responses monitored at 1 pg/mL, 10 pg/mL, 100 pg/mL and 1 ng/mL antibody concentrations in PBS;

FIG. 4C is a plot of real-time responses monitored at 1 pg/mL, 10 pg/mL, 100 pg/mL and 1 ng/mL antibody concentrations in human whole blood; and

FIG. 4D is a graph showing a comparison between the plots of normalized responses at each antibody concentration in PBS and human whole blood, in nanograms per milliliter, with the graph usable as a calibration curve for interpolating an S1 protein IgG antibody concentration given a measured response.

### DETAILED DESCRIPTION

The detailed description of exemplary embodiments makes reference to the accompanying drawings, which show exemplary embodiments by way of illustration and their best mode. While these exemplary embodiments are described in sufficient detail to enable those skilled in the art to practice the invention, it should be understood that other embodiments may be realized and that logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In accordance with various embodiments of the present disclosure, a biosensor is configured to detect both an antigen and an antibody specific to the antigen in a sample. In various embodiments, the biosensor comprises an array of In₂O₃ nanoribbon FET devices disposed on a chip.

### Definitions

TABLE 1 sets forth brief definitions of various terms used throughout.

TABLE 1
Definitions

| Term | Description |
|---|---|
| FET | Field Effect Transistor |
| In₂O₃ | Indium Oxide |
| LoD | Limits of detection |
| Iₒₙ | On-state current (maximum current at a constant drain |
| | voltage) |
| I_{off} | Off-state current (the minimum current at the same drain |
| | voltage) |
| (Iₒₙ/I_{off}) | The ratio of on-state current to off-state current |
| I/I₀ | Measured current divided by baseline current |
| I/I₀ (%) | Measured current divided by baseline current as a |
| | percentage |
| I_{sd} | The current between the source and the drain electrodes |
| | in a FET |
| V_{liquid gate} | The voltage applied by the liquid gate electrode to the |
| | liquid medium |
| (I_{DS} − V_{GS}) | Source to drain current versus applied gate voltage |
| Average | Average electrical mobility in (cm²/V · s) |
| mobility | |

### GENERAL EMBODIMENTS

In various embodiments, a biosensor comprises an array of In₂O₃ nanoribbon FET devices disposed on a chip. The biosensor is configured to detect an antigen in a sample provided to the biosensor. In other configurations, the biosensor is capable of detecting antibodies specific to an antigen in a sample provided to the biosensor.

In various embodiments, a biosensor capable of detecting both SARS-CoV-2 antigen and antibody is described. Further, methods of using a biosensor for detecting SARS-CoV-2 antigen and antibody are described.

In various embodiments, a biosensor capable of detecting both SARS-CoV-2 S1 protein and SARS-CoV-2 S1 protein IgG antibody is described, recognizing the spike (S) protein is on the SARS CoV-2 viral envelope and its S1 subunit is the outermost component of the virus. Further, methods of using a biosensor for detecting SARS-CoV-2 S1 protein and SARS-CoV-2 S1 protein IgG antibody are described.

In various embodiments, a biosensor capable of detecting both SARS-CoV-2 S1 protein and SARS-CoV-2 S1 protein IgG antibody comprises a plurality of nanoribbon field-effect transistor (FET) devices. In various embodiments, each nanoribbon comprises indium oxide (In₂O₃).

In various embodiments, the nanoribbon of each FET device of the biosensor is configured as a channel, having a channel length, a channel width, and a channel thickness.

In various embodiments, each FET device of the biosensor comprises a substrate with a source electrode disposed thereon and a drain electrode also disposed on the substrate and spaced apart from the source electrode by a gap configured as a channel. The source and drain electrodes are electrically connected by a In₂O₃ nanoribbon in the channel and spanning the gap.

In various embodiments, each of the source and drain electrode pairs in the biosensor comprise titanium (Ti) and/or gold (Au).

In various embodiments, the biosensor further comprises a substrate, such as Si/SiO₂ substrate, beneath the source electrode, the drain electrode, and the nanoribbon channel. In various embodiments, the Si/SiO₂ substrate provides a structure onto which the electrodes and the nanoribbon are deposited in a fabrication process comprising shadow masking.

In various embodiments, the biosensor further comprises a solution gate electrode and is configured as a solution-gated FET (SGFET). In various embodiments, the gate electrode comprises a silver/silver chloride (Ag/AgCl) electrode immersed in a solution (a liquid medium) that encloses each of the pairs of source and drain electrodes and associated nanoribbons of a biosensor. Thus configured, each FET device in an array of devices is a solution-gated FET device.

In various embodiments, a liquid gate voltage (V_{liquid gate}) is applied to a biosensor by the solution gate electrode immersed in the liquid medium surrounding the biosensor. A liquid gate bias is applied and changes in a current of the device is measured as an amperometric signal as various changes occur to the liquid medium, such as a pH of the medium.

In various embodiments, each FET device of a biosensor further comprises at least one capture antibody conjugated to the nanoribbon of the FET, wherein the antibody specifically targets a biomarker whose presence is to be identified. In certain aspects, this conjugation comprises a chemical linker that provides bonding between the indium oxide (In₂O₃) nanoribbon and the capture antibody. In various aspects, there may be varying numbers of antibodies immobilized on each of the nanoribbons of a biosensor herein, such as at least one antibody, and in some instances, none.

In various embodiments, the at least one capture antibody specifically targets S1 antigen of SARS-CoV-2. In various embodiments, the biosensor configured with the SARS-CoV-2 S1 protein IgG antibody as the capture antibody is capable of detecting the SARS-CoV-2 S1 antigen protein in both phosphate-buffered saline (PBS buffer) and universal transport medium (UTM), wherein biotinylated secondary antibody is incubated with streptavidin-conjugated phosphatase enzyme to form an enzyme-antibody conjugate, and the resulting conjugate associates with the biosensor.

In various embodiments, the biosensor is capable of being switched from S1 antigen detection to SARS-CoV-2 spike protein IgG antibody detection by adding a step of antigen incubation and changing the secondary antibody conjugated to the phosphatase to anti-human IgG antibody.

In various embodiments, a plurality of nanoribbon FET devices are configured in an array on a chip, such as a CMOS chip, which once functionalized with the appropriate molecules, becomes a biosensor.

The SARS-CoV-2 Virus

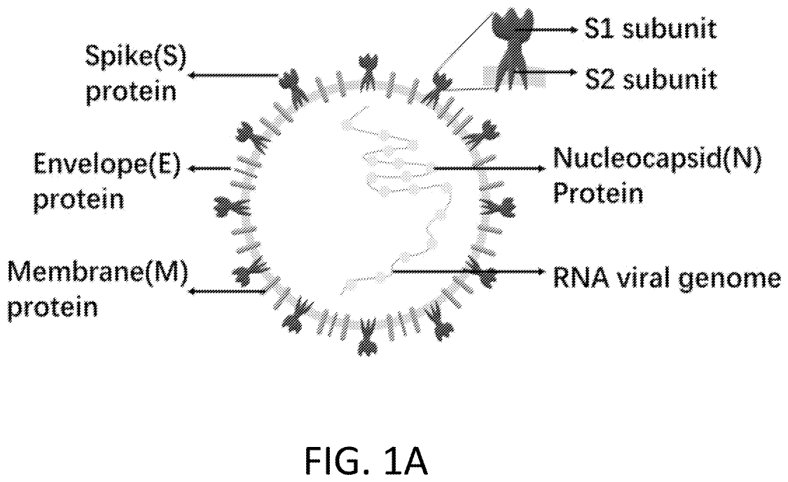

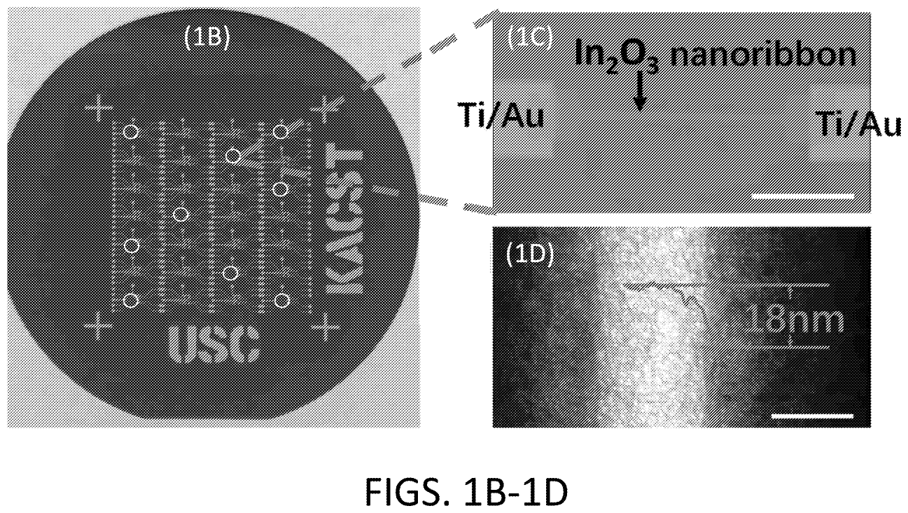

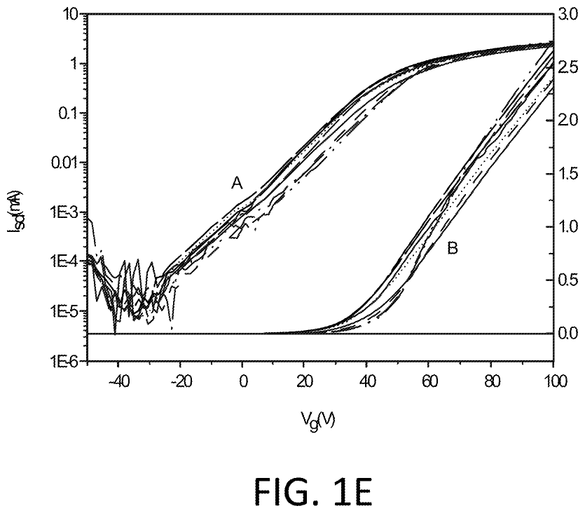

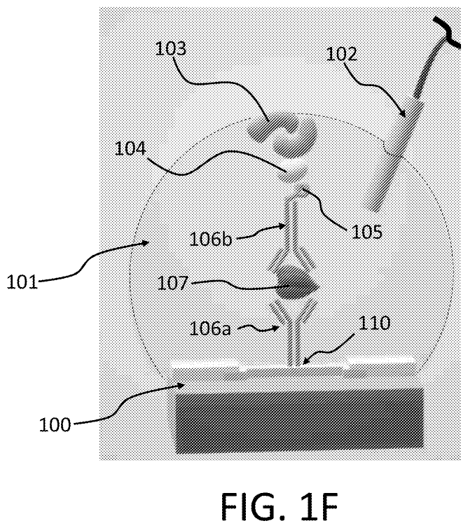

With reference now to FIG. 1A, SARS-CoV-2 virus mainly consists of four types of proteins, namely spike(S), membrane(M), nucleocapsid(N), and envelop(E) proteins. Among these proteins, the S protein is a trimer consisting of HR2, HR1 and S1 subunits. The S protein is on the viral envelope and its S1 subunit (referred to herein more simply as the “S1 protein”) is the outermost component of the virus. During SARS-CoV-2 infection, S1 proteins recognize and bind with cellular receptors on the surface of the target cells, facilitating the virus particles to enter the target cells and trigger an infection. Correspondingly, S1 protein is an important target for SARS-CoV-2 diagnostic detection. Moreover, detecting S1 protein specific antibody in human blood is also important for COVID-19 diagnosis in terms of determining previous viral exposure and immunity status, such as following vaccine treatment. Antibody tests (also known as serology test) can be nearly 100% accurate for blood samples collected 20 days after infection or onset of symptoms.

Indium Oxide Biosensors

The present disclosure describes a versatile electronic biosensing platform capable of detecting both SARS-CoV-2 S1 protein and S1 protein IgG antibody, whose detection target can be easily switched between the two biomarkers.

Among recent advances in rapid screening methods, field-effect transistor (FET)-based biosensors have many advantages, such as, for example, small size, real-time detection, high-sensitivity, and capability for integrated multiplexing. Graphene-based FET biosensors for detecting the spike protein of SARS-CoV-2 have been described, while others demonstrated single-walled carbon nanotube (SWCNT)-based FETs to probe spike and nucleocapsid proteins of SARS-CoV-2.

Graphene is semi-metallic while semiconducting carbon nanotubes usually exhibit p-type conductivity. To reduce the probability of getting false positive and false negative diagnoses, it is desirable to have complementary sensing capability by using both p-type and n-type semiconductors. Indium oxide (In₂O₃) is an n-type semiconductor successfully applied as the channel material for a variety of biosensors. However, in accordance with the present disclosure, In₂O₃ transistors have now been fabricated using a lithography-free process. This simplified fabrication method make In₂O₃ biosensors cost-efficient and suitable for large-scale COVID-19 clinical diagnosis.

FET-based biosensors can be used in conjunction with enzymatic reactions because many enzymatic reactions release acidic or basic products resulting in a pH change of the environment to generate electronic signals of the FETs. Although urease is the most common enzyme incorporated with FET-based biosensors, urease displays poor stability, losing most of its enzymatic activity after being stored at 4° C. for only 10 days. In accordance with the present disclosure, alkaline phosphatase is applied as the enzyme reporter for the electronic biosensors herein. During the sensing process, alkaline phosphatase produces protons by enzymatically removing a phosphate group from the substrate compound, and the decreased pH value of the surrounding medium will then lead to an increase in the current of the inventive In₂O₃ FETs. Previously, phosphatase has been used as the enzyme in commercial colorimetric ELISA kits (LoD: 16 pg/mL, 0.55 mg/mL). Compared to urease, phosphatase is much more stable and suitable for long-term storage while maintaining high activity. Consequently, the present biosensing platform with phosphatase overcomes inherent limits to electronic biosensing, whose application was previously constrained by the poor stability of the enzyme reporter. The present biosensors, on the other hand, display a prolonged shelf life and are correspondingly advantageous for practical COVID-19 diagnosis.

In₂O₃ biosensors in accordance with the present disclosure were able to detect S1 antigen of SARS-CoV-2 in both phosphate-buffered saline (PBS) buffer and universal transport medium (UTM) with a reliable performance and similar LoD of 100 fg/mL. Besides antigen detection, S1 protein IgG antibody detection in both PBS and human whole blood (WB) were also carried out. The detection performance in both mediums displayed LoD of 1 pg/mL. The similar detection results in different mediums indicate the capability of the present electronic biosensors to eliminate the interference of salts, cells, and other particles in detected specimens. Taken together, the present biosensors are highly sensitive and scalable, showing good potential for clinical SARS-CoV-2 diagnosis. These findings shed light on a new generation of biosensors for rapid and highly sensitive COVID-19 screening, clearly a potential win in the fight against COVID-19 pandemic.

### Experimental Methods

1. Materials

3-inch Si/SiO₂ (500 nm) wafers were purchased from UniversityWafer, Inc., Boston, Mass. Shadow masks for patterning were purchased from Photo Sciences, Inc., Redlands, Calif. Au and Ti metal sources for electron-beam evaporation and an indium oxide (In₂O₃) sputtering target with purity of 99.99% were obtained from Plasmaterials, Inc., Livermore, Calif. 3-Phosphonopropionic acid with a purity of 94%, EDC with a purity of 98%, NHS with a purity of 98%, 10% BSA, phosphatase substrate, and streptavidin conjugated alkaline phosphatase were each purchased from Sigma-Aldrich. 2019-nCoV Spike Protein S1 (40591-V08H) and SARS-CoV-2 spike antibody (40150-D001 and 40150-D003) were purchased from Sino Biological, Inc., Chesterbrook, Pa. Biotin conjugated Anti-Human IgG was obtained from Abcam, Cambridge, England. UTM and swab collection kits were purchased form COPAN Diagnostics Inc., Murrieta, Calif. Human whole blood was obtained from Innovative Research, Inc., Novi, Mich.

2. Shadow Mask Fabrication Method

A SiO₂/Si wafer was first rinsed with acetone and isopropyl alcohol, followed by a baking process at 120° C. for 5 min to remove all solvent residuals. The wafer was then cooled at room temperature. After the cleaning/drying process, the first shadow mask was attached to the SiO₂/Si wafer and a plurality of In₂O₃ nanoribbons were then deposited by radio frequency (RF) sputtering (by Denton Vacuum Discovery 550 sputtering system in NRF) to pattern the channel area. Subsequently, pairs of source and drain electrodes (1 nm Ti and 50 nm Au, deposited by electron-beam evaporation) were defined by a second shadow mask, wherein a single pair of source and drain electrodes were deposited at and over the ends of each nanoribbon such that the source and drain electrodes in each pair of source and drain electrodes were electrically connected by a single In₂O₃ nanoribbon. The result is an array of FET devices disposed on a substrate, each FET device comprising spaced apart Ti/Au source and drain electrodes electrically connected by a In₂O₃ nanoribbon.

Further details regarding the method of fabrication of biosensors for use herein and in accordance with the present disclosure are found in U.S. Patent Application Publication No. 2019/0120788, C. Zhou, et al., published Apr. 25, 2019, incorporated herein by reference in its entirety for all purposes.

3. Functionalization of In₂O₃ Devices

In₂O₃ devices were immersed in boiling acetone and isopropyl alcohol for 5 minutes each to clean the surface, followed by an O₂ (150 mTorr, 100 W, 40 s) plasma treatment. After that, the devices were immersed in 0.1 mM aqueous 3-phosphonopropionic acid for 16 hours before annealing at 120° C. for 12 hours in an Argon flow. Next, devices were treated with a mixture of 40 mM EDC and 30 mM NHS in DI water for 1 hour.

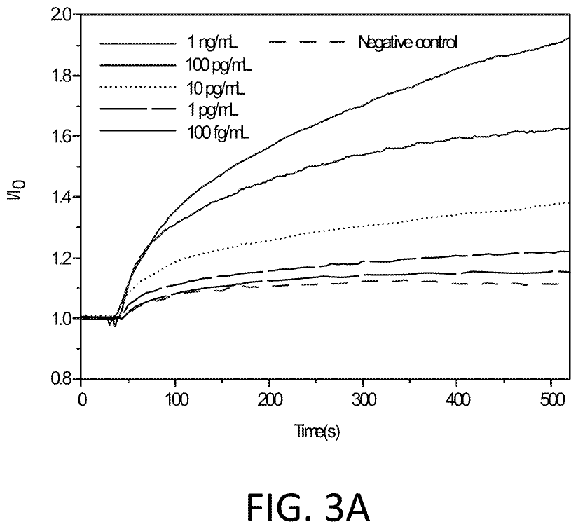

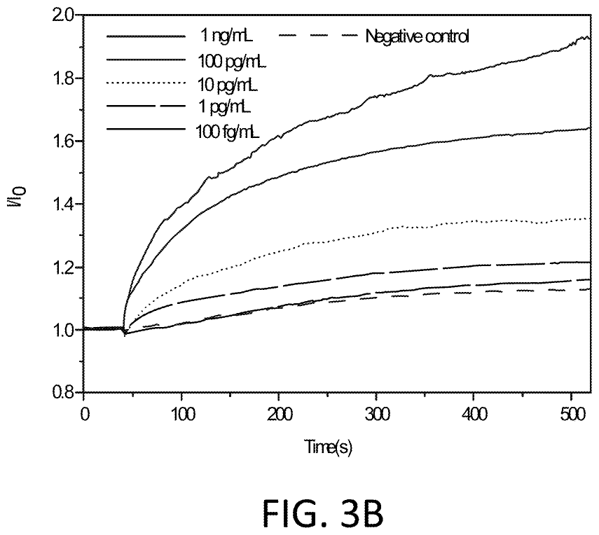

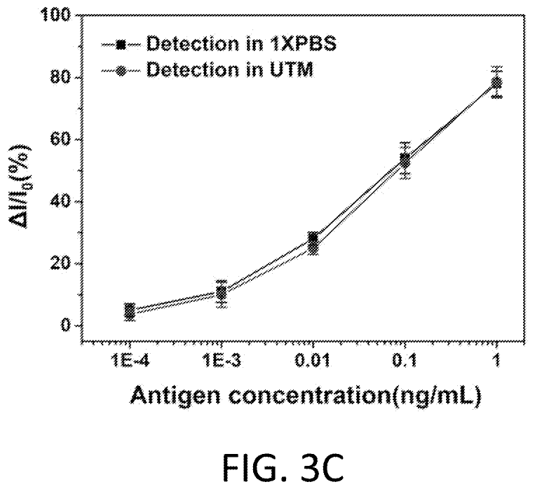

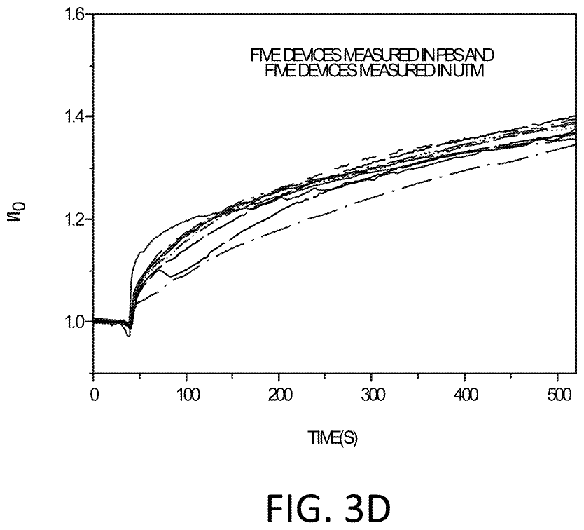

For S1 protein detection, devices were then incubated in 50 μg/mL SARS-CoV-2 spike antibody in PBS for 1 hour, target S1 protein in PBS (or UTM) for 1 hour, 50 μg/mL biotin conjugated secondary antibody in PBS for 1 hour, and 150 μg/mL streptavidin conjugated phosphatase in PBS for 40 min, consecutively. Known concentrations of target S1 protein in either medium are used to generate a calibration curve (e.g., FIG. 3C) such that an amperometric signal obtained from a sample of unknown S1 protein concentration can be interpolated on the calibration curve to determine a corresponding S1 protein concentration. In various embodiments, the presence of the target S1 antigen on the device is detected when a reactive solution is applied to the device.

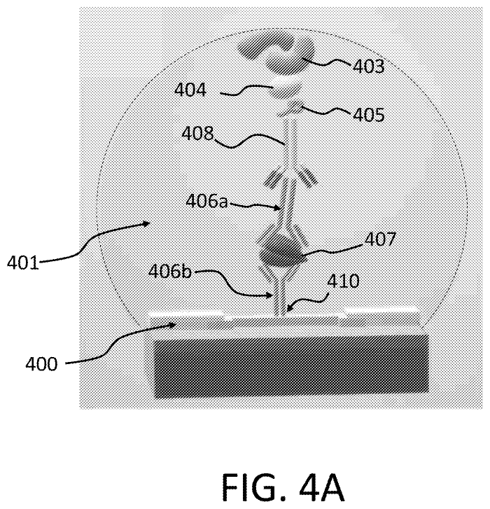

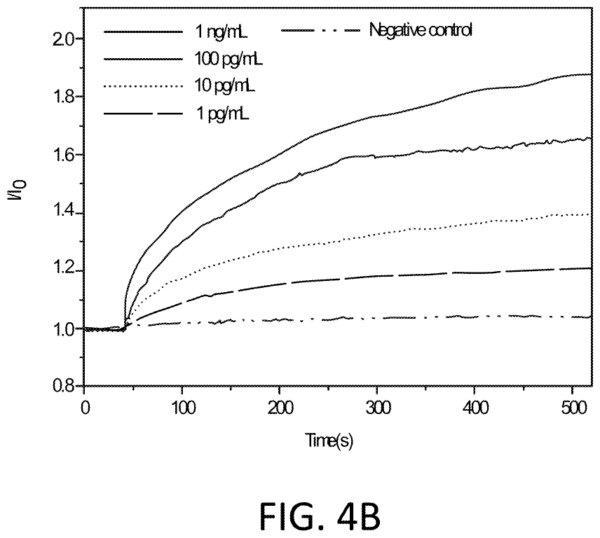

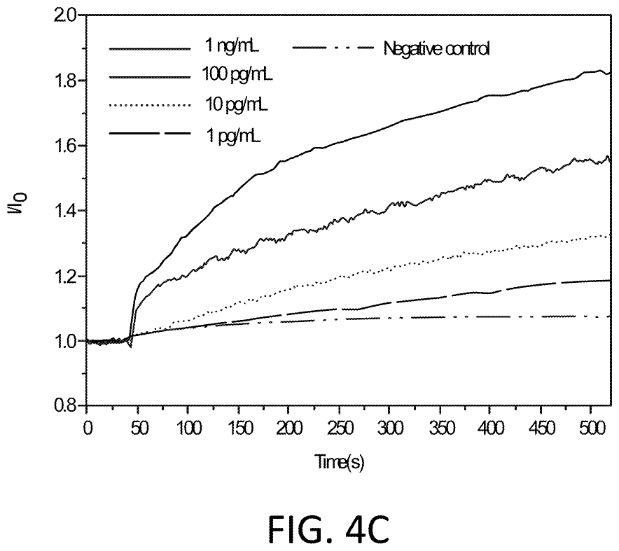

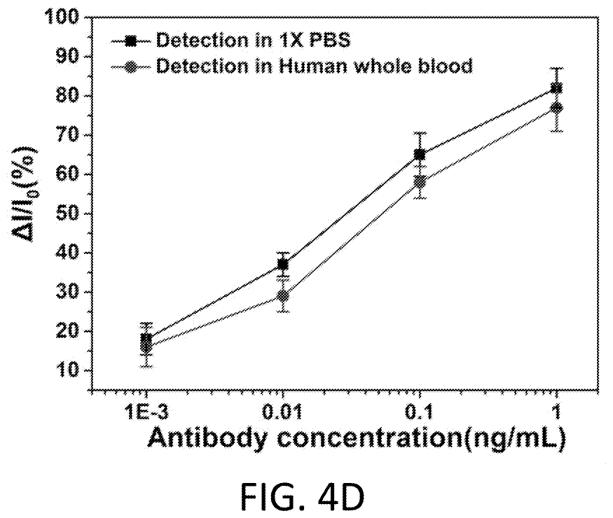

For antibody detection, devices were incubated in 50 μg/mL SARS-CoV-2 spike antibody in PBS for 1 hour, 15 μg/mL S1 protein in PBS for 1 hour, target antibody in PBS (or human whole blood, or other medium to provide for a biosample) for 1 hour, 50 μg/mL biotin conjugated secondary antibody for 1 hour, and 150 μg/mL streptavidin conjugated phosphatase in PBS for 40 min, consecutively. Known concentrations of target antibody in either medium can be used to generate a calibration curve (e.g., FIG. 4D) such that an amperometric signal obtained from a sample of unknown antibody concentration can be interpolated on the calibration curve to determine a corresponding antibody concentration. In various embodiments, the presence of the SARS-CoV-2 spike antibody on the device is detected when a reactive solution is applied to the device.

4. Characterization

Optical microscopy images were taken with an Olympus microscope. Atomic force microscopy (AFM) imaging was performed on a Digital Instruments DI 3100. Electrical characteristics of the In₂O₃ FETs in ambient environment and sensing results were measured with an Agilent 1500B semiconductor analyzer.

Results are discussed below in context with the various graphs provided in the drawing figures.

Biosensors Based on In₂O₃ Nanoribbon FET Devices

FIG. 1B is a photograph of a plurality of In₂O₃ nanoribbon FET devices configured on a Si/SiO₂ (500 nm) wafer substrate, in accordance with various procedures of the present disclosure. In other examples, a wafer substrate may comprise silicon, PET or glass.

The lithography-free fabrication of the FETs comprised the two steps detailed above and further detailed in US2019/0120788 incorporated herein by reference. First, In₂O₃ nanoribbons were defined by a first shadow mask and deposited onto a substrate using radio frequency (RF) sputtering. As shown by the magnified image of FIG. 1C, the channel length and channel width of the In₂O₃ nanoribbons thus produced were 500 μm and 25 μm, respectively. The scale bar in FIG. 1C is 200 μm. FIG. 1D shows an Atomic Force Microscopy (AFM) image of an individual nanoribbon of one FET, with the channel thickness thus determined to be about 18 nm. The scale bar in FIG. 1D measures 20 μm. Next, pairs of source and drain electrodes were defined by apertures in a second shadow mask. After alignment of the second shadow mask, 1 nm Ti and 50 nm Au were deposited by electron beam evaporation to form a pair of source and drain electrodes on the ends of each of the nanoribbons. A plurality of twenty-eight biosensing chips can be fabricated on a 3-inch substrate, with each chip comprising an array of 4 separate In₂O₃ FET devices. As shown in the photograph of FIG. 1B, the chip pattern in this fabricated example is a 4×7 array, totaling 28 chips (one hundred and twelve (112) devices on a wafer).

The electrical performance of the devices was first characterized in ambient environment using an Agilent Semiconductor Analyzer 1500B. Nine different In₂O₃ FET devices were randomly selected across the substrate for analysis, (the locations for analysis are shown in FIG. 1B by the nine open white circles drawn over the photograph), and their drain current versus back gate voltage (I_{DS}−V_{GS}) curves with the drain voltage fixed at 1V are shown as plots in FIG. 1E, the bundle of plots labeled (a) being linear and the bundle of plots labeled (b) being logarithmic. The I_{DS}−V_{GS} curves of the FET devices thus measured illustrate n-type transistor behavior with an average mobility of 108±4.3 cm²/V·s and on-off ratio of ˜1×10⁵. Owing to the thick SiO₂ dielectric, the devices can be turned on only at high back gate voltage. As evident from FIG. 1E, the nine individual plots of this graph nearly superimpose, demonstrating an electrical uniformity of the In₂O₃ nanoribbon devices over the entire wafer.

FIG. 1F is a schematic diagram of an individual In₂O₃ device capable of detection of SARS-CoV-2 S1 protein in accordance with various aspects of the present disclosure. This device structure is the result of the fabrication procedures and conjugation steps detailed above and corresponds to the structure of an individual functionalized FET device on the wafer of FIG. 1B.

With continued reference to FIG. 1F, a Teflon fluidic cell with an opening at the bottom was used to contain a liquid medium 101 and ensure contact of the medium 101 with each of the In₂O₃ biosensors 100 across the entire wafer. The fluidic cell encloses the biosensor such that the array of FET devices are placed in contact with any solution provided to the cell, or any solution provided to the fluidic cell in combination with a liquid medium already present in the cell. As discussed in the experimental procedures herein above, prior to electronic biosensing, the In₂O₃ devices 100 were first immersed in boiling acetone and isopropyl alcohol for 5 minutes each to clean the surface, followed by an O₂ plasma treatment to generate hydroxyl groups on the surface of In₂O₃. The linker 3-phosphonopropionic acid was then reacted with the hydroxyl groups provided on the surface. Subsequently, devices were functionalized with N-(3-dimethylaminopropyl)-N′-ethylcarbodiimide hydrochloride/N-hydroxysuccinimide (EDC/NHS), creating a plurality of activated carboxyl groups capable of binding with amino groups on proteins. Next, capture antibodies 106a that specifically target S1 antigens of SARS-CoV-2 were applied and subsequently immobilized onto the In₂O₃ nanoribbons 110 via reaction with the EDC/NHS groups previously functionalized on the surface. This immobilization process is followed by a washing step to rinse off unbound capture antibodies 106a as well as a blocking step with 10% bovine serum albumin (BSA) solution to deactivate the surface of the In₂O₃ in order to avoid non-specific binding during consecutive processes. Target biomarker S1 antigens 107 were then introduced to the biosensors. In various examples, S1 antigens may be present in a biosample (based on a buffer or other preparation such as PBS or UTM) provided to the biosensor for analysis. S1 antigen solutions with known concentrations were dissolved in either PBS or UTM followed by a 1-hour incubation period. This resulted in the binding of S1 antigen 107 to the anchored capture antibodies 106a. Next, biotinylated secondary antibodies 106b were introduced and incubated for 1 hour, followed by a 40-minute incubation of streptavidin conjugated phosphatase. The resulting structure, as shown in FIG. 1F, comprises the S1 antigen 107 bound between two antibodies, 106a and 106b, with the biotin 105 of the biotinylated secondary antibody 106b binding the phosphatase enzyme 103 through streptavidin 104. Finally, phosphatase substrate solution was applied (in the medium 101) and an amperometric signal recorded as the result of the detection. The total time from application of the biomarkers (i.e., functionalization) to the time of obtaining a final sensing result is nearly 3 hours, with actual detection time ranging from 10 minutes to 15 minutes.

Electronic Biosensing Realized by Enzymatic Reaction of Phosphatase

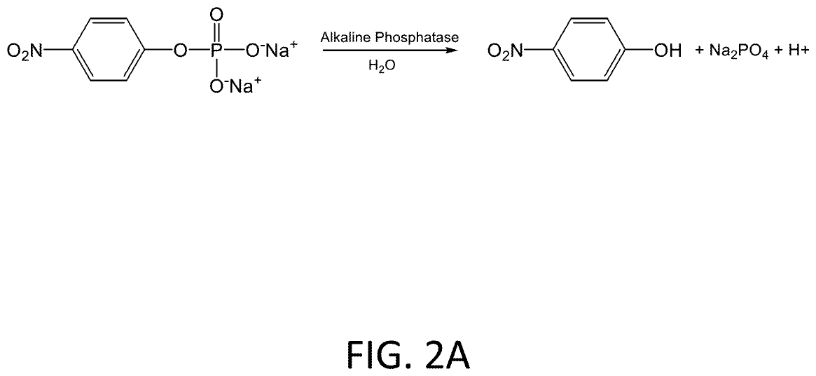

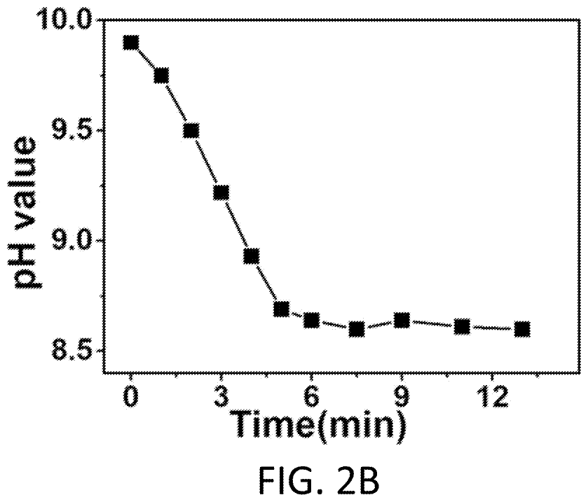

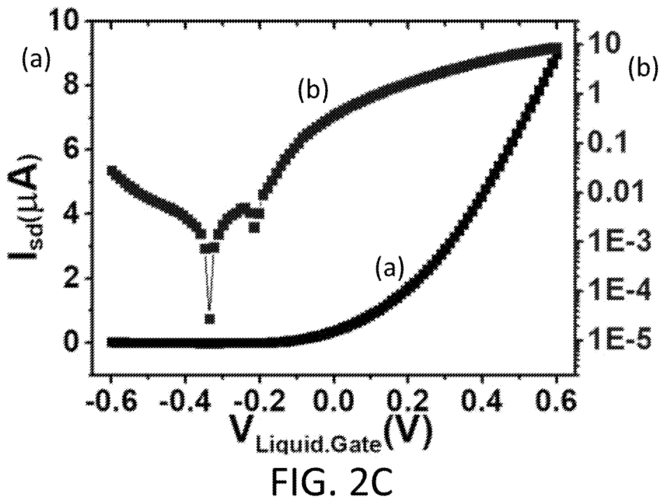

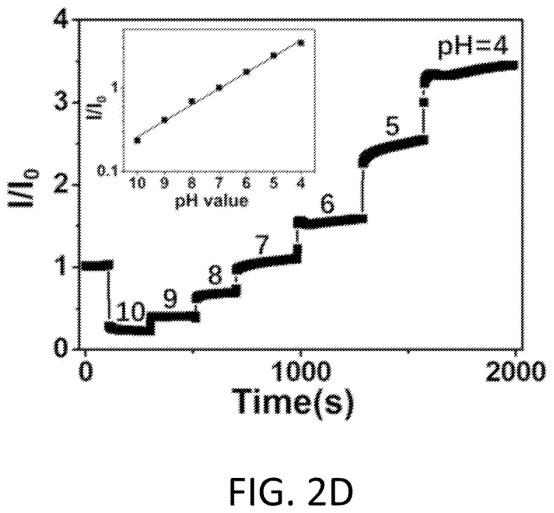

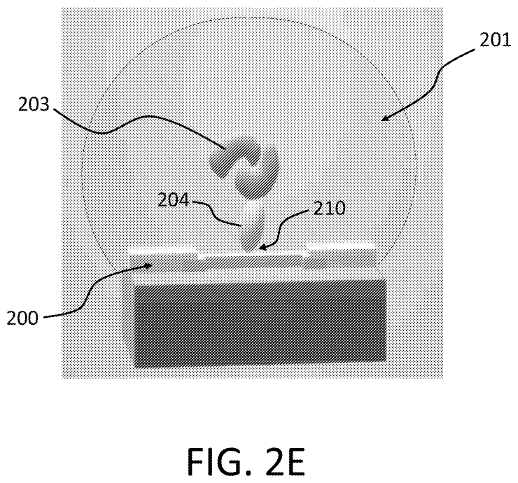

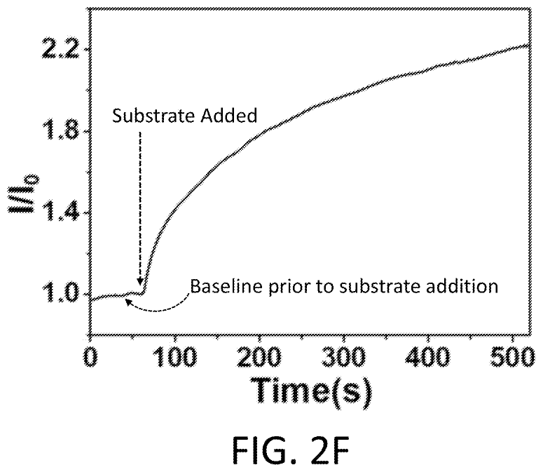

In various aspects of the present disclosure, electronic detection of SARS-CoV-2 is based on enzymatic reaction of alkaline phosphatase. As shown by the chemical reaction scheme in FIG. 2A, when catalytic alkaline phosphatase is present, the phosphate group of a substrate compound is removed, producing protons that lead to a change in the pH of the medium surrounding each FET device. To prepare the substrate solution, 10 mg of 4-nitrophenyl phosphate disodium salt hexahydrate, (the commercial phosphatase substrate typically used for colorimetric ELISA), was dissolved in 10 mL of 0.5M MgCl₂ solution. The pH of the substrate solution was then adjusted to 9.7 by addition of dilute NaOH solution. Change in pH was monitored after manually mixing 5 μL of 1.5 mg/mL alkaline phosphatase solution with 3 mL of the substrate solution. As shown in the plot of FIG. 2B, the pH decreased by about 1.3 over the course of 5 minutes, with a concomitant color change of the solution from colorless to bright yellow.

As demonstrated, the In₂O₃ nanoribbon FET devices served as a pH sensor in response to a pH change caused by the phosphatase enzymatic reaction. Hence, devices require a wet environment (as shown in FIG. 1F, the wet environment provided by the surrounding medium 101 in a Teflon cell). An Ag/AgCl reference electrode (102 in FIG. 1F) was used to apply a liquid gate bias. FIG. 2C shows the drain current versus liquid gate voltage (I_{DS}−V_{GS}) curve of the devices immersed in 0.01×PBS buffer with the drain voltage fixed at 200 mV. A corresponding family of curves of drain current versus drain voltage (I_{DS}−V_{DS}) showed that In₂O₃ FETs display good current saturation at high V_{DS}. Based on these measurements, it was deduced that the In₂O₃ FETs in accordance with the present disclosure can be reliably and efficiently controlled using the liquid gate with on/off ratios (Iₒₙ/I_{off}) of 10⁴.

With reference now to FIG. 2D, when the enzymatic reaction starts, the deprotonated hydroxyl groups on the surface of In₂O₃ nanoribbons become protonated as the pH decreases. Thus, as shown in the inset in FIG. 2D, the negative charges on the surface of In₂O₃ nanoribbons decrease and the conduction of the material increases. FIG. 2D also shows the current change of the FET devices in response to pH change, (with a fixed liquid gate voltage of 150 mV and a fixed drain voltage of 200 mV), showing that the I_{DS} of the device increased by a factor of 14 as the pH dropped from 10 to 4.

In order to demonstrate that In₂O₃ nanoribbon FET devices and phosphatase enzymatic reactions can be integrated for electronic biosensing, a positive control experiment was conducted with devices configured as schematically illustrated in FIG. 2E. The streptavidin-conjugated phosphatase (phosphatase enzyme 203 conjugated to streptavidin 204) was directly attached to the nanoribbon 210 of the FET device 200 by EDC/NHS chemistry, while the detailed incubation procedure was similar to the S1 antigen detection procedure described above.

With reference now to FIG. 2F, to ensure detection of the final amperometric signal, prior to adding substrate solution, the channel region of the In₂O₃ FET was immersed in a MgCl₂ and NaOH solution having the same pH as the substrate solution. A liquid gate voltage of 150 mV was applied while the drain voltage was fixed at 200 mV in order to generate the observed baseline shown in FIG. 2F. Substrate solution was then added (at the point shown in FIG. 2F) to trigger the enzymatic reaction, and the I_{DS} of the device was recorded as a function of time. As shown in FIG. 2F, the I_{DS} started to increase as soon as the substrate was introduced, resulting in a current enhancement of 110% after about 8 minutes. This real-time monitoring from the positive control setup demonstrates phosphatase is compatible with the electronic biosensing technique of the present disclosure.

Detection of SARS-CoV-2 Spike Protein in PBS and UTM

Electronic biosensing targeting the S1 antigen of the SARS-CoV-2 spike protein was performed in 1×PBS with known concentrations of the S1 protein ranging from 100 fg/mL to 1 ng/mL.

FIG. 3A shows the normalized responses for each S1 protein concentration of 100 fg/mL, 1 pg/mL, 10 pg/mL, 100 pg/mL and 1 ng/mL in PBS, with 4%, 10%, 26%, 50% and 80% observed current increases, respectively (after subtracting a negative control). The negative control was obtained by merely replacing the S1 antigen biomarker solution with BSA solution during the functionalization process. The slight increase in the signal of the negative control might be caused by non-specific binding of phosphatase to the In₂O₃ nanoribbons.

The detection of S1 protein dissolved in UTM with the same choices of S1 protein concentrations as per the BSA solutions was also carried out. UTM, or universal transport medium, is a common solvent for collection, transport, maintenance, and long-term freeze storage of clinical specimens containing viruses. It's widely used for cell culture, rapid antigen detection, PCR, and nucleic acid amplification assays. Hence, antigen detection in UTM helps to assess the present biosensor to be used for clinical diagnosis of SARS-CoV-2.

In order to simulate a real clinical setting, a nasopharyngeal swab sample taken from a healthy person was used in a UTM-based detection setup. The swab was suspended in UTM along with S1 protein to produce exemplary biosamples. FIG. 3B shows the obtained sensing signal where 4%, 8%, 23%, 52%, and 80% current changes were observed in response to antigen concentrations of 100 fg/mL, 1 pg/mL, 10 pg/mL, 100 pg/mL and 1 ng/mL S1 protein in UTM, respectively.

FIG. 3C show a plot of both PBS and UTM responses. The results of detections in PBS (square icons) and UTM (circle icons) are plotted in FIG. 3C. The results illustrate that the ingredients of UTM, such as Hank's balanced salts, BSA and sucrose, do not interfere with the sensing results, since the results in UTM are virtually identical to the results in PBS. This finding reveals that the present electronic biosensing approach can circumvent Debye screening from salts in the fluid, indicating that the present biosensors can be used for clinical diagnostic testing without any special sample preparation or pretreatment.

The LoD of the present biosensor reached 100 fg/mL, which is 2 orders of magnitude lower than conventional colorimetric ELISA. This could be caused by the local reaction nature of the present sensing technique. It is expected that localized pH changes of the solution immediately surrounding the In₂O₃ nanoribbon generate the observed amperometric signals, rather than changes in the pH of the entire solution, the latter of which would require a greater number of phosphatase enzyme molecules anchored to the surface of In₂O₃ and a correspondingly prolonged period of time for detection. This hypothesis is supported by taking amperometric measurements while bubbling the solutions extensively to mix thoroughly. When doing so, the current dropped back to a point close to baseline, indicating the pH of the solution overall did not change appreciably. Once the bubbling is ceased, the current increased again as the enzymatic reaction changed the local pH of the solution immediately adjacent the In₂O₃ nanoribbons.

To investigate reproducibility of this detection method, and in particular the reproducibility of the FET device fabrication, biosensing of 10 pg/mL S1 antigen in different mediums was conducted using devices obtained from 4 different fabrication batches. As shown in FIG. 3D, 3 rounds of detection in PBS and 1 round of detection in UTM generated similar sensing responses across fabrication batches. Hence, fabricated biosensors in accordance with the present disclosure exhibit a remarkable reproducibility for large scale clinical diagnostic testing.

Detection of Spike Protein IgG Antibody in PBS and in Human Whole Blood

To demonstrate the versatility of the present electronic biosensing platform, the In₂O₃ devices were employed in the detection of SARS-CoV-2 spike protein IgG antibody. As mentioned, antibody detection is important for identifying past infections and assessing viability of vaccines.

FIG. 4A schematically shows the structure of a surface functionalized In₂O₃ nanoribbon configured for Si spike protein specific IgG antibody detection and used herein for proof of this concept. In this structure, the sequence of molecule binding along with the conjugated antibody and S1 protein serve as the capturing component. As shown in the illustration, capture antibodies 406a that specifically target S1 antigens of SARS-CoV-2 are immobilized onto the In₂O₃ nanoribbons 410. Secondary target antibodies 406b are bound to the capture antibodies 406a via by the S1 antigen 407. Biotinylated Anti-Human IgG antibody (i.e., biotinylated anti-SARS-CoV-2 IgG antibody to spike protein Si) 408 is introduced as the secondary antibody followed by the introduction of streptavidin-conjugated phosphatase 404/403 that binds to the bound Anti-Human IgG antibody 408 by the biotin 405. This structure is used to record amperometric signals upon introduction of substrate solution with the device is immersed in a liquid gate setup having the surrounding medium 401.

FIG. 4B shows the amperometric signals generated as the result of detecting antibodies dissolved in PBS buffer solution. 1 pg/mL, 10 pg/mL, 100 pg/mL and 1 ng/mL concentrations of antibodies generated 17%, 36%, 61%, and 84% of current enhancement, respectively, as shown by the plots in the graph. It's worth noting that the anti-Human IgG antibody could potentially bind with the primary antibody. Therefore, to investigate this possibility and/or effect of this undesired binding, a negative control experiment was performed where BSA solution was used as replacement of the target antibody solution. FIG. 4B shows the result of this negative control, where a 4% current increase was generated. This indicates that the anti-Human IgG antibody does not appreciably bind with the primary antibody anchored to the surface of In₂O₃, and as such, does not appreciably affect the sensing results.

In various aspects of the present disclosure, the biosensors exhibit high selectivity when detecting S1 protein specific antibody in physiological solutions. In this experiment, target antibodies at known concentrations were prepared in human whole blood (WB) and used in detection experiments. S1 protein specific antibody concentration was varied to include 1 pg/mL, 10 pg/mL, 100 pg/mL and 1 ng/mL concentrations, which led to current enhancements of 16%, 30%, 54% and 80%, respectively shown in the plots of FIG. 4C.

A comparison of the results in PBS and human whole blood is provided in FIG. 4D. In this graph, detection in 1×PBS is the plot with square icons and the detection in human whole blood is the plot with circle icons. As evident from the plots in FIG. 4D, antibodies in PBS antibodies in human whole blood (WB) generated analogous responses while the magnitude of sensing responses detecting antibody in WB being slightly lower than that in PBS. These miniscule differences could stem from the higher viscosity of whole blood compared to PBS, where precipitated blood cells can attach to the surface of the biosensor during the incubation period, and thus could induce interference with the sensing results. Nevertheless, it can be concluded that the sensing behaviors in both mediums are mainly attributed to the binding of the target antibody.

The results of detecting SARS-CoV-2 S1 antigen in PBS and UTM, and S1 protein specific antibody in PBS and human whole blood reveal that the present In₂O₃ devices and electronic biosensing approach provide highly sensitive, dual-functional sensing for COVID-19 diagnosis. The ability of the present biosensors to be switched from antigen detection to antibody detection by simply adding an additional step of antigen incubation and changing the secondary antibody, along with their high immunity to interference caused by ions, cells, and other particles from complex fluids, makes the present biosensors cost-efficient and desirable for worldwide rapid screening of SARS-COV-2 virus with high sensitivity.

The demonstrated electronic biosensing platform based on phosphatase and shadow mask fabrication process is not limited to In₂O₃ material and SARS-CoV-2 biosensing. This technology has the potential to be applied to other semiconducting materials such as silicon nanowires, metal oxide nanowires/nanoribbons, carbon nanotubes, graphene, and other two-dimensional materials for biosensing applications. In addition, the electronic biosensing technique based on phosphatase has the potential to be generalized as a standard protocol of electronic biosensors for highly sensitive detection of various proteins with prolonged shelf life.

Since the enzyme and other reagents used in the functionalization of the present biosensors are also used for commercial colorimetric ELISA kits, it is possible to integrate the present biosensors with conventional colorimetric ELISA for a dual-readout biosensor platform. Such integration would improve the sensing characteristics with high reliability. In this platform, colorimetric signal can be used for quick and preliminary detection, while the electrical signal can subsequently be used for a more sensitive diagnosis.

In conclusion, sensitive, scalable, and cost-efficient COVID-19 biosensors using electronic biosensing platform based on In₂O₃ FET devices functionalized with phosphatase is now demonstrated. The devices were fabricated by a simple and inexpensive shadow mask method. The present biosensors were able to detect SARS-CoV-2 spike protein in UTM (LoD: 100 fg/mL) and S1 protein specific IgG antibody in human whole blood (LoD: 1 pg/mL), indicating its potential for clinical diagnostic testing. These results can be instrumental for the management and control of the ongoing and possible future pandemics and can possibly prevent further community transfer through early, rapid, and cost-efficient screening of COVID-19, giving an upper hand in pandemics.

### ADDITIONAL CONSIDERATIONS

In various embodiments, a biosensor comprises an array of In₂O₃ FET devices configured on a substrate, the array of In₂O₃ FET devices being fluidically accessible by a fluidic cell enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, and wherein at least some of the In₂O₃ nanoribbons in the array of In₂O₃ FET devices include at least one capture antibody immobilized thereon, the capture antibodies being specific to an antigen.

In various embodiments, the capture antibodies immobilized on the nanoribbons are specific to an antigen of interest. In various examples, the biosensor is used to analyze for the presence of the antigen of interest in a sample, or to determine a concentration of the antigen of interest in the sample. A liquid sample is provided to the fluidic cell, and hence the sample is fluidically placed into contact with the array of In₂O₃ FET devices of the biosensor. In various examples, the fluidic cell may begin with a liquid medium (such as PBS or UTM) and the liquid medium is then diluted with solutions added at various times. In other examples, a solution to be placed into contact with the array of In₂O₃ FET devices of the biosensor is provided to the fluidic cell with concomitant displacement of the prior contents in the fluidic cell.

In various embodiments, the capture antibodies are specific to the S1 spike protein antigen of SARS-CoV-2 virus. In various examples, the biosensor is used to analyze for the presence of the SARS-CoV-2 S1 antigen in a sample, or to determine a concentration of the SARS-CoV-2 S1 antigen in the sample. A liquid sample is provided to the fluidic cell, and hence the sample is fluidically placed into contact with the array of In₂O₃ FET devices of the biosensor.

In various embodiments, a method of detecting the presence of or a concentration of an antigen in a sample comprises:

fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibody being specific to the antigen, and wherein the antigen binds to the immobilized capture antibody;

fluidically contacting biotinylated secondary antibodies with the array of In₂O₃ FET devices of the biosensor, the biotinylated secondary antibodies being specific to the antigen, wherein the biotinylated secondary antibody binds to the antigen previously bound to the immobilized capture antibody;

fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation;

fluidically contacting alkaline phosphatase substrate with the array of In₂O₃ FET devices of the biosensor, the alkaline phosphatase substrate being capable of enzymatic cleavage by the alkaline phosphatase, wherein the alkaline phosphatase cleaves the alkaline phosphatase substrate, producing protons and a pH change in the liquid medium; and detecting an amperometric signal from the biosensor as a result of the pH change in the liquid medium, indicating a presence of the antigen in the sample.

In various embodiments, the method is used to determine a concentration of the antigen in the sample by interpolation on a calibration curve. In certain examples, the calibration curve comprises a x/y plot of current change in the biosensor (ΔI/I₀ (%)) versus antigen concentration. In this way, a detected amperometric signal, such as a current change in the biosensor (ΔI/I₀ (%)), is interpolated on the calibration curve to an antigen concentration.

In various embodiments, the amperometric signal comprises a current in the biosensor.

In various embodiments, the amperometric signal comprises a current change in the biosensor.

In various embodiments, the amperometric signal comprises a percent current change (ΔI/I₀ (%)) in the biosensor.

In various embodiments of the method, the alkaline phosphatase substrate comprises disodium p-nitrophenyl phosphate.

In various embodiments, a method of detecting the presence of or a concentration of SARS-CoV-2 S1 antigen in a sample comprises:

fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibody being specific to SARS-CoV-2 S1 antigen, and wherein the SARS-CoV-2 S1 antigen binds to the immobilized capture antibody;

fluidically contacting biotinylated secondary antibodies with the array of In₂O₃ FET devices of the biosensor, the biotinylated secondary antibodies being specific to SARS-CoV-2 S1 antigen, wherein the biotinylated secondary antibody binds to the SARS-CoV-2 S1 antigen previously bound to the immobilized capture antibody;

fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation;

fluidically contacting alkaline phosphatase substrate with the array of In₂O₃ FET devices of the biosensor, the alkaline phosphatase substrate being capable of enzymatic cleavage by the alkaline phosphatase, wherein the alkaline phosphatase cleaves the alkaline phosphatase substrate, producing protons and a pH change in the liquid medium; and detecting an amperometric signal from the biosensor as a result of the pH change in the liquid medium, indicating a presence of SARS-CoV-2 S1 antigen in the sample.

In various embodiments, the method is used to determine a concentration of SARS-CoV-2 S1 antigen in the sample by interpolation on a calibration curve. In certain examples, the calibration curve comprises a x/y plot of current change in the biosensor (ΔI/I₀ (%)) versus SARS-CoV-2 S1 antigen concentration. In this way, a detected amperometric signal, such as a current change in the biosensor (ΔI/I₀ (%)), is interpolated on the calibration curve to a SARS-CoV-2 S1 antigen concentration.

In various embodiments, the amperometric signal comprises a current in the biosensor.

In various embodiments, the amperometric signal comprises a current change in the biosensor.

In various embodiments, the amperometric signal comprises a percent current change (ΔI/I₀ (%)) in the biosensor.

In various embodiments of the method, the alkaline phosphatase substrate comprises disodium p-nitrophenyl phosphate.

In various embodiments, a method of detecting the presence of or a concentration of an antibody in a sample specific to an antigen comprises:

fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibodies being specific to and having antigen bound thereto, and wherein the antibodies in the sample bind to the antigens bound to the immobilized capture antibodies;

fluidically contacting biotinylated secondary antibodies with the array of In₂O₃ FET devices of the biosensor, the biotinylated secondary antibodies comprising anti-human antibody to the antigen, wherein the biotinylated secondary antibodies bind to the antibodies previously bound to the antigens immobilized on the capture antibodies;

fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation;

fluidically contacting alkaline phosphatase substrate with the array of In₂O₃ FET devices of the biosensor, the alkaline phosphatase substrate capable of enzymatic cleavage by the alkaline phosphatase, wherein the alkaline phosphatase cleaves the alkaline phosphatase substrate, producing protons and a pH change in the liquid medium; and

detecting an amperometric signal from the biosensor as a result of the pH change in the liquid medium, indicating a presence of the antibodies in the sample.

In various embodiments, the antibody in the sample comprises SARS-CoV-2 spike protein IgG antibody and the antigen to which the antibody is specific to comprises SARS-CoV-2 S1 antigen.

In various embodiments, the method is used to determine a concentration of antibodies in the sample by interpolation on a calibration curve. In certain examples, the calibration curve comprises a x/y plot of current change in the biosensor (ΔI/I₀ (%)) versus antibody concentration. In this way, a detected amperometric signal, such as a current change in the biosensor (ΔI/I₀ (%)), is interpolated on the calibration curve to an antibody concentration.

In various embodiments, the amperometric signal comprises a current in the biosensor.

In various embodiments, the amperometric signal comprises a current change in the biosensor.

In various embodiments, the amperometric signal comprises a percent current change (ΔI/I₀ (%)) in the biosensor.

In various embodiments of the method, the alkaline phosphatase substrate comprises disodium p-nitrophenyl phosphate.

In various embodiments, a method of detecting the presence of or a concentration of SARS-CoV-2 spike protein IgG antibodies in a sample comprises:

fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibodies being specific to and having SARS-CoV-2 S1 antigen bound thereto, and wherein the SARS-CoV-2 spike protein IgG antibodies in the sample bind to the S1 antigens of SARS-CoV-2 bound to the immobilized capture antibodies;

fluidically contacting biotinylated secondary antibodies with the array of In₂O₃ FET devices of the biosensor, the biotinylated secondary antibodies comprising anti-human SARS-CoV-2 IgG antibody to spike protein S1, wherein the biotinylated secondary antibodies bind to the SARS-CoV-2 spike protein IgG antibodies previously bound to the S1 antigens immobilized on the capture antibodies;

fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation;

fluidically contacting alkaline phosphatase substrate with the array of In₂O₃ FET devices of the biosensor, the alkaline phosphatase substrate capable of enzymatic cleavage by the alkaline phosphatase, wherein the alkaline phosphatase cleaves the alkaline phosphatase substrate, producing protons and a pH change in the liquid medium; and

detecting an amperometric signal from the biosensor as a result of the pH change in the liquid medium, indicating a presence of the SARS-CoV-2 spike protein IgG antibodies in the sample.

In various embodiments, the method is used to determine a concentration of SARS-CoV-2 spike protein IgG antibodies in the sample by interpolation on a calibration curve. In certain examples, the calibration curve comprises a x/y plot of current change in the biosensor (ΔI/I₀ (%)) versus SARS-CoV-2 spike protein IgG antibody concentration. In this way, a detected amperometric signal, such as a current change in the biosensor (ΔI/I₀ (%)), is interpolated on the calibration curve to a SARS-CoV-2 spike protein IgG antibody concentration.

In various embodiments, the amperometric signal comprises a current in the biosensor.

In various embodiments, the amperometric signal comprises a current change in the biosensor.

In various embodiments, the amperometric signal comprises a percent current change (ΔI/I₀ (%)) in the biosensor.

In various embodiments of the method, the alkaline phosphatase substrate comprises disodium p-nitrophenyl phosphate.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A method of detecting the presence of, or a concentration of, an antigen in a sample, the method comprising:
fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibody being specific to the antigen, and wherein the antigen binds to the immobilized capture antibody;
fluidically contacting biotinylated secondary antibodies with the array of In₂O₃ FET devices of the biosensor, the biotinylated secondary antibodies being specific to the antigen, wherein the biotinylated secondary antibody binds to the antigen previously bound to the immobilized capture antibody;
fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation;
fluidically contacting alkaline phosphatase substrate with the array of In₂O₃ FET devices of the biosensor, the alkaline phosphatase substrate being capable of enzymatic cleavage by the alkaline phosphatase, wherein the alkaline phosphatase cleaves the alkaline phosphatase substrate, producing protons and a pH change in the liquid medium; and
detecting an amperometric signal from the biosensor as a result of the pH change in the liquid medium, indicating a presence of the antigen in the sample.

2. The method of claim 1, wherein the pH change in the liquid medium comprises a pH decrease resulting in an increase in conductivity of each In₂O₃ nanoribbon.

3. The method of claim 1, further comprising determining a concentration of the antigen in the sample by interpolating the amperometric signal thus detected on a calibration curve that relates amperometric signal to antigen concentration.

4. The method of claim 3, wherein the calibration curve comprises an x/y plot of percent current change in the biosensor ((ΔI/I₀ (%)) versus antigen concentration.

5. The method of claim 1, wherein the antigen comprises SARS-CoV-2 S1 antigen.

6. The method of claim 1, wherein the alkaline phosphatase substrate comprises disodium p-nitrophenyl phosphate.

7. The method of claim 1, wherein the biosensor further comprises a solution gate electrode in fluidic contact with the liquid medium such that each In₂O₃ FET device is configured as a solution-gated FET.

8. The method of claim 7, further comprising applying a liquid gate voltage to the solution gate electrode prior to detecting an amperometric signal comprising a current or change in current in the biosensor.

9. The method of claim 1, wherein each In₂O₃ nanoribbon is characterized by a channel length about 500 μm, a channel width of about 25 μm, and a channel thickness of about 18 nm.

10. A method of detecting the presence of, or a concentration of, an antibody in a sample specific to an antigen, the method comprising:
fluidically contacting the sample with an array of In₂O₃ FET devices of a biosensor, the array of In₂O₃ FET devices configured on a substrate and fluidically accessible by a fluidic cell containing a liquid medium enclosing the biosensor, each In₂O₃ FET device comprising a source electrode disposed on the substrate, a drain electrode disposed on the substrate, spaced apart from the source electrode, and a In₂O₃ nanoribbon disposed on the substrate and electrically connecting the source electrode and the drain electrode, wherein each In₂O₃ nanoribbon in the array of In₂O₃ FET devices is defined by a channel width, channel length and channel thickness, wherein each In₂O₃ nanoribbon in the array of In₂O₃ FET devices includes at least one capture antibody immobilized thereon, the capture antibody being specific to and having the antigen bound thereto, and wherein the antibodies in the sample bind to the antigens bound to the immobilized capture antibodies;
fluidically contacting biotinylated secondary antibodies with the array of In₂O₃ FET devices of the biosensor, the biotinylated secondary antibodies comprising anti-human antibody to the antigen, wherein the biotinylated secondary antibodies bind to the antibodies previously bound to the antigens immobilized on the capture antibodies;
fluidically contacting streptavidin alkaline phosphatase conjugate with the array of In₂O₃ FET devices of the biosensor, wherein the alkaline phosphatase binds to the biotinylated secondary antibody through a streptavidin-biotin conjugation;
fluidically contacting alkaline phosphatase substrate with the array of In₂O₃ FET devices of the biosensor, the alkaline phosphatase substrate capable of enzymatic cleavage by the alkaline phosphatase, wherein the alkaline phosphatase cleaves the alkaline phosphatase substrate, producing protons and a pH change in the liquid medium; and
detecting an amperometric signal from the biosensor as a result of the pH change in the liquid medium, indicating a presence of the antibodies in the sample.

11. The method of claim 10, wherein the antibody in the sample comprises SARS-CoV-2 spike protein IgG antibody and the antigen to which the antibody is specific to comprises SARS-CoV-2 S1 antigen.

12. The method of claim 10, further comprising determining a concentration of the antibodies in the sample by interpolating the amperometric signal thus detected on a calibration curve that relates amperometric signal to antibody concentration.

13. The method of claim 12, wherein the calibration curve comprises an x/y plot of percent current change in the biosensor ((ΔI/I₀ (%)) versus antibody concentration.

14. The method of claim 10, wherein the alkaline phosphatase substrate comprises disodium p-nitrophenyl phosphate.

15. The method of claim 10, wherein the biosensor further comprises a solution gate electrode in fluidic contact with the liquid medium such that each In₂O₃ FET device is configured as a solution-gated FET.

16. The method of claim 15, further comprising applying a liquid gate voltage to the solution gate electrode prior to detecting an amperometric signal comprising a current or change in current in the biosensor.

17. The method of claim 10, wherein each In₂O₃ nanoribbon is characterized by a channel length about 500 μm, a channel width of about 25 μm, and a channel thickness of about 18 nm.

18. The method of claim 10, wherein the pH change in the liquid medium comprises a pH decrease resulting in an increase in conductivity of each In₂O₃ nanoribbon.
